(19) United States
(12) Patent Application Publication
(10) Pub. No.: US 2012/0022376 A1
(43) Pub. Date: Jan. 26, 2012

(54) METHOD AND A SYSTEM FOR MEDICAL IMAGING

(75) Inventors: Arie Amara, Misgav (IL); Avi Amara, Doar-Na Menashe (IL)

(73) Assignee: Helix Medical Systems Ltd., Kiryat-Shmona (IL)

(21) Appl. No.: 13/201,499

(22) PCT Filed: Feb. 1, 2010
(86) PCT No.: PCT/IL2010/000085

§ 371 Date: Oct. 6, 2011

Related U.S. Application Data
(60) Provisional application No. 61/202,291, filed on Feb. 13, 2009.

(51) Int. Cl. A61B 8/08

(52) U.S. Cl. 600/443; 600/459

(57) ABSTRACT

A device configured for performing a spatial scan of a patient's breast. The device comprises a substantially hollow housing having a substantially conical surface between a top and a substantially hollow base. The substantially hollow housing is sized and shaped for covering at least a portion of a breast. The device further comprises a scanning device for performing a scan while being maneuvered in a scanning pattern adjacent to the substantially conical surface. The distance between the top and the substantially hollow base is less than 5 centimeters.

### FIELD AND BACKGROUND OF THE INVENTION

The present invention, in some embodiments thereof, relates to a system and a method for medical imaging and, more particularly, but not exclusively, to a system and a method for diagnosis and treatment of breast tumors.

Ultrasound tomography uses sound waves to create two- or three-dimensional images of anatomical structures and avoids the use of ionizing radiation inherent with other forms x-ray mammography and PET based imaging. Ultrasound is also more cost efficient then Breast MRI. Ultrasound sonography is widely used in the medical community as tools for diagnosis and sometimes even in therapy. Sonographers commonly use hand-held probes that transmit and receive ultrasonic waves radiated into the region of interest of a patient. A water-based gel is typically used to couple the probe to the patient's skin. Ultrasound is effective for imaging soft tissues. High frequencies (7-18 MHz) are typically used to image shallow structures, while lower frequencies (1-6 MHz) having poorer spatial resolution but better penetration are typically used to image structures deeper in the body.

The success of ultrasound as a method for detecting breast lesions is highly dependent on the skill of the sonographer. Reducing dependence on skilled sonographers may facilitate its effective application to large numbers of patients and improve the workflow. Automated scanning is a technique that may allow more user independent sonographic imaging. Automated scanning has the benefit of being reproducible in scan quality, location, and orientation, thus allowing comparison to previous imaging and enhance the workflow by “decoupling” the data acquisition part from the diagnostic part of the diagnostic process. Reproducible scans are useful in clinical situations for comparison to previous results and where a need exists to evaluate the progress of cancer treatment Automatic three-dimensional scans could provide important 3 D presentation of the region of interest and help the surgeon to find the exact location of tumor. thus supporting more targeted surgery (M. Halliwell, S. Curtis; Visions Magazine 2008 No. 12).

In recent years, advances in ultrasound scanning have been made in several areas. U.S. Pat. No. 4,206,763 filed Aug. 1, 1978 describes an apparatus comprising a compartment in which water is drawn upward by suction over the breast being examined, and in which an ultrasonic transducer revolves around the breast to achieve complete 360° scans. U.S. Pat. No. 4,455,872 filed Apr. 25, 1983 describes a rotating scanner for use in ultrasonic echoscopy that has a linearly scanned transducer arrangement. The transducer arrangement is rotated about an axis passing through the center or one end of the linear scan. Rotational movement of the transducer's scanning plane achieves visualization over a volume within the object under examination. International Patent Application Publication No. WO03/103500, filed Jun. 2, 2003, describes a device for use in the imaging of breast tissue comprising a mounting structure capable of holding an ultrasound transducer having an effective transmission face and a tissue molding element for receiving and surrounding the breast tissue. The tissue molding element has a three-dimensional shape that is approximately conical, domed, or ellipsoidal segment. The relative movement of the mounting structure takes place in two dimensions over the three-dimensional surface of the breast tissue placed into the tissue molding element.

U.S. patent application Ser. No. 11/513,481 filed Aug. 30, 2006 describes an apparatus and related methods for facilitating volumetric ultrasonic scanning of a breast. A generally cone-shaped radial scanning template having a vertex and a wide opening angle is provided, the radial scanning template having a slot-like opening extending outward from the vertex through which an ultrasonic transducer scans the breast as the radial scanning template is rotated. The template compresses the breast and rotates.

In U.S. patent application Ser. No. 10/559,078 filed Mar. 15, 2005, Amara, et al. (henceforth: “Amara”) describes a portable mechanical high-precision device for performing circular or helical scanning of a patient's organ or body surface for tissue diagnosis and/or treatment. Discussion from this point until the beginning of the summary refers to Amara. The device includes a substantially hollow housing to accommodate the organ and a securing unit for securing the housing to the organ or body surface during scanning. At least one drive unit is attached to the housing and to at least one scan-head to allow unlimited rotation of the scan-head relative to the housing. For purposes of better understanding some embodiments of the present invention, reference is first made to general and exemplary construction and operation of Amara.

Reference is now made to FIG. 1 which pictorially shows a substantially conical housing 10 for accommodating a breast (not shown) during scanning. Reference is also made to FIG. 2, which is a schematic illustration showing the principle components in a device for scanning a patient's breast tissue. The housing 10 has a first end 11 having a threaded portion 12 for threadably attaching to a motor unit so as to allow rotation of the housing. Apertures 13 formed in the first end 11 of the housing cooperate with a vacuum pump (not shown) for allowing suction to be applied. The apertures 13 thus operate as a securing unit for securing the breast within the housing and thereby avoid dislocation thereof during scanning, which if not prevented would derogate from the accuracy of subsequent measurements. A second end 15 of the housing is open to allow for insertion of the breast and may be provided with a circumferential flange 16 for adding strength and rigidity.

The device 30 in FIG. 2 comprises a first motor 31 adapted to rotate the housing 10 via a worm gear drive 32. To this end, the threaded portion 12 in the first end 11 of the housing 10 is connected via a complementary threaded portion (not shown) to one end of a coupler unit (concealed in the figure) whose other end is coupled to the worm gear drive 32. The coupler unit is surrounded by an outer sleeve 33 that is free to rotate relative to the housing 10 and is provided at an end remote from the housing with a peripheral coupling 34 that is rotatably coupled to a second motor 35. A bracket assembly 36 is fixed to the sleeve 33 via a pair of bolts such as 37 and supports a linear motor 38 (constituting a third motor) that is articulatedly coupled to a scan-head assembly 39 accommodating therein in an ultrasound probe 40. In such an arrangement, the first motor 31 is adapted to rotate the housing 10 only about an axis A, while the second motor 35 is adapted to rotate the scan-head assembly 39 together with the linear motor 38 relative to the housing 10 also about the about the axis A. Either during such rotation of the scan-head assembly 39 or independent thereof, the scan-head assembly may be moved linearly up and down the outer wall of the housing 10 in the direction of B-B along a line parallel to an axis thereof. By such means, the scan-head assembly is amenable to three types of motion: circular motion by driving the second motor 35 only; linear motion by driving the linear motor 38 only; and helical motion by driving both motors 35 and 38 simultaneously.

During initial scan mode of operation, acoustic gel is applied to the breast which is then placed within an appropriately sized housing and secured therein by applying suction. The acoustic gel improves coupling when using an ultrasound scan-head. After the breast has been secured within the housing, the motors for rotational and linear movement are operated simultaneously under control of the computer to produce circular scanning of the scan-head assembly. Scan data are stored in computer memory and/or storage media. After completion of the scan cycle, the data is analyzed by known methods, typically in the absence of the patient. An example of analysis is described in Digital Picture Processing, 2nd edition 1982, Azriel Rosenfeld, K. C. Avinash, ISBN 0-12-597302-0, Academic Press, Inc.

### SUMMARY OF THE INVENTION

According to some embodiments of the present invention there is provided a device configured for performing a spatial scan of a patient's breast, including

a substantially hollow housing having a substantially conical surface between a top and a substantially hollow base, the substantially hollow housing being sized and shaped for covering at least the center of the breast,

a scanning device configured for performing a scan while being maneuvered in a scanning pattern adjacent to the substantially conical surface,

a securing unit for securing the substantially hollow housing to the breast during the scan so that the breast being substantially fixed relative to the housing, and

wherein a distance between the top and the substantially hollow base is less than 5 centimeters.

Optionally, the housing is substantially domed, conical, polyhedral, or ellipsoidal.

Optionally, the ratio between the distance and a diameter of the substantially hollow base is less than 58.5.

Optionally, the ratio between the distance and a diameter of the substantially hollow base is less than 326.

Optionally, the substantially hollow housing is sized and shaped for applying substantially uniform extrinsic direct linear pressure perpendicular to at least 80 percent of the surface of the patient's breast.

Optionally, the substantially conical surface is slanted at an angle of less than 30 degrees to a plane concurrent with the substantially hollow base.

Optionally, the distance between the top and the substantially hollow base is less than 3 cm.

Optionally, holes penetrate the substantially conical surface through which vacuum pressure is applied inside the substantially hollow housing.

Optionally, the device further includes a flexible extender affixed to the housing adjacent to the substantially hollow base to increase the volume of the housing.

Optionally, the housing further includes a liquid bath in which the scanning device performs a spatial scan of a patient's breast.

Optionally, the scanning device is positionable at a plurality of angles relative to the housing.

Optionally, the housing includes an inflatable balloon inside the substantially hollow housing, the balloon being configured to compress the tissue of the breast in an inflated configuration.

Optionally, the housing consists essentially of polymethylpentene.

Optionally, the device includes a mechanical arm having at least 4 degrees of freedom.

Optionally, the mechanical arm further includes a piston configured to compress a breast of a patient during scanning.

Optionally, the device further includes a table for enabling a patient to lie in a stable prone position, wherein the table enables the patient's breast to hang dependent through an opening in the table, and the securing unit secures the breast during the scan.

According to some embodiments of the present invention there is provided a device configured for performing a spatial scan of a patient's breast, including

a substantially hollow housing having a substantially conical surface between a top and a substantially hollow base,

a scanning device configured for performing a scan while being maneuvered adjacently to the substantially conical surface, and

an inflatable balloon positioned in the substantially hollow housing and configured for being inflated by a ultrasound conducting liquid before the scan.

Optionally, the housing further includes an aperture sized and shaped for facilitating insertion of a tool for at least one of diagnosis and treatment.

According to some embodiments of the present invention there is provided a method of performing ultrasound imaging including,

transmitting a plurality of distinct ultrasound frequencies into breast tissue,

intercepting scattered signals of the distinct ultrasound frequencies to create a plurality of images from the breast tissue, and

combining the plurality of images to create an image representing the breast tissue.

Optionally, the imaging includes tomography.

According to some embodiments of the present invention there is provided a method of performing ultrasound imaging including,

placing the patient's breast into a substantially hollow housing and securing the breast therein,

compressing the tissue of the breast such that the depth of the tissue from the top to the base of the housing is less than 5 centimeters, and

scanning the breast with a scanning device.

Optionally, the scanning includes maneuvering the scanning device adjacent to the housing in a substantially rotational or helical scanning pattern.

Optionally, the compressing includes inflating a balloon that is enclosed in the housing with ultrasound conducting liquid.

Optionally, the method further includes filling a bath with ultrasound-conducting liquid, submerging the scanning device in the liquid and scanning the breast while the scanning device is submerged in the liquid.

Optionally, the method further includes positioning the scanning device at a plurality of angles relative to the breast.

Optionally, the method further includes affixing, configuring, and aligning a flexible extender to the housing to modify the area of the breast that is available for scanning.

Optionally, the securing includes the application of vacuum pressure to the inside of the housing through holes in the surface of the housing.

According to some embodiments of the present invention there is provided a method for scanning a body organ, including

providing a probe,

mounting a housing around a body organ,

positioning a container including an ultrasound conducting material between the housing and the probe, and

maneuvering the probe adjacently to the housing for performing a scan of at least a portion of the body organ while the ultrasound conducting material being in physical contact with the housing and the probe.

Optionally, the container is a bath for holding the ultrasound conducting material,

the mounting including fully or partially submerging the probe in the ultrasound conducting material.

Optionally, the container is a holder containing a replaceable gel pad, and the positioning includes attaching the gel pad to the probe, such that during the scanning the pad physically interfaces between the probe and the housing. Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings and/or images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic illustration that pictorially shows a substantially conical housing for accommodating a breast during scanning;

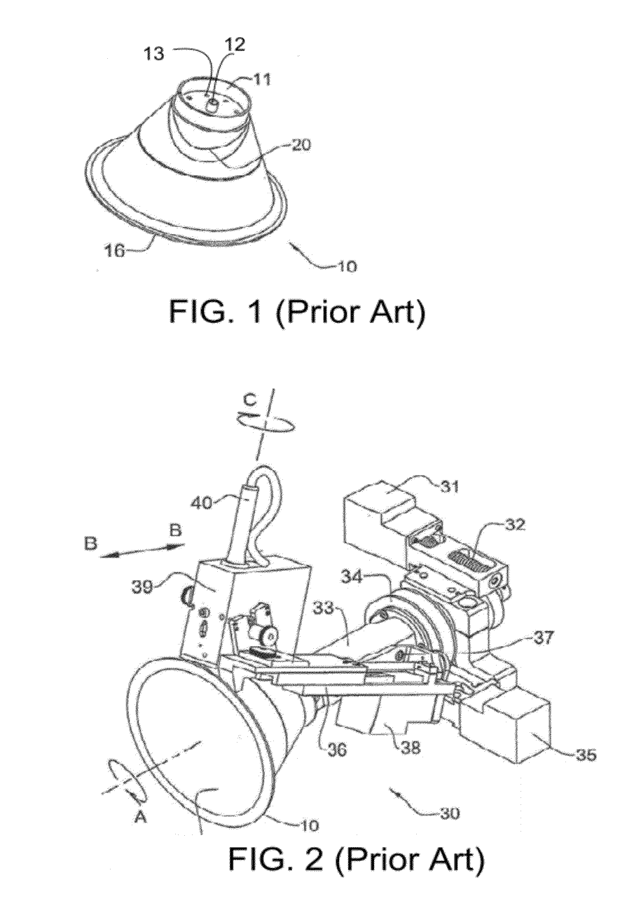

FIG. 2 is a schematic illustration showing the principle components in a device for scanning a patient's breast tissue;

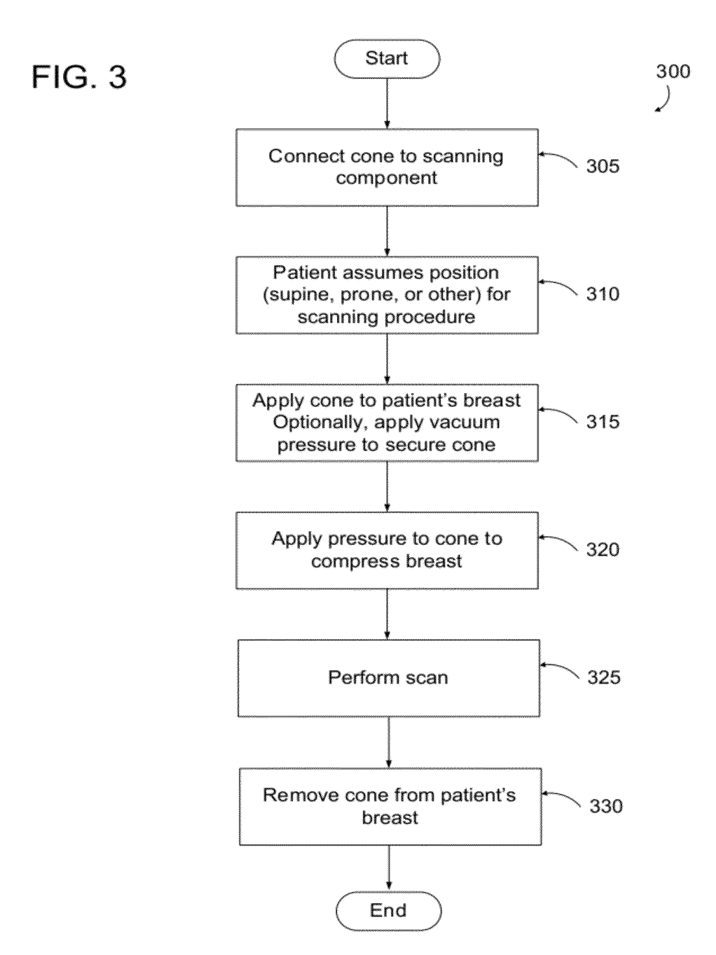

FIG. 3 is a flowchart describing the how a breast-flattening housing may be used according to some embodiments of the present invention;

FIGS. 4A-4D are a schematic illustrations of an exemplary breast-flattening housing wherein FIG. 4A is a sectional view through a diameter, FIG. 4B is sectional view of a housing wall at the open end, FIG. 4C is a bottom view of the housing, and FIG. 4D is a view of a threaded element and holes for vacuum application, according to some embodiments of the present invention;

FIGS. 5A-5B are illustrations of an exemplary breast-flattening housing, according to some embodiments of the present invention;

FIGS. 5C-5D are photographs of an exemplary breast-flattening housing, according to some embodiments of the present invention;

FIG. 6 is an image of an ultrasound breast scan exhibiting a black hole phenomenon;

FIG. 7A-7B are exemplary breast-flattening housings with a volume extending attachment;

FIGS. 8A-8C are tables showing measurements of housings made of TPX, according to some embodiments of the present invention;

FIG. 9 is an illustration representing using the device with a patient in an oblique-supine position, according to some embodiments of the present invention;

FIG. 10A is an illustration representing using the device with a patient in a prone position on a table built to enable access of the device to the patient's breast and/or breasts from below, according to some embodiments of the present invention;

FIG. 10B and FIG. 10C which are photographs of a table built to enable access of the device to the patient's breast and/or breasts from below while the patient is in a prone position, according to some embodiments of the present invention;

FIG. 11 is an illustration representing a gel pad holder, according to some embodiments of the present invention;

FIG. 12 is an illustration representing an ultrasound probe with the gel pad holder of FIG. 11, according to some embodiments of the present invention;

FIGS. 13A-13D are a schematic diagram of a fluid-bath comprising a breast-flattening housing for ultrasound imaging according to some embodiments of the present invention; and

FIG. 14 is an illustration of a liquid-bath used for ultrasound imaging, according to some embodiments of the present invention.

### DESCRIPTION OF SPECIFIC EMBODIMENTS OF THE INVENTION

The present invention, in some embodiments thereof, relates to a system and a method for medical imaging and, more particularly, but not exclusively, to a system and a method for diagnosis of breast lesions and treatment of breast tumors.

According to some embodiments of the present invention, the device is indicated for medical imaging of a patient's breast wherein the breast is substantially uniformly flattened and/or compressed against the chest wall such that the breast's tissues are limited in depth, for example, to less than 4 centimeters from a scanning probe during an ultrasound scan. Automatic scanning may obtain multiple sequential two-dimensional or 3D images that may be compiled into a complete or partial three dimensional dataset for viewing. The system may automatically acquire a complete or partial breast image and may generate multi-planar reconstruction (MPR) slices or other form of 2D or 3D presentation of the scanned breast. The system may produce breast images by acquiring in a circular imaging sequence or in other geometric orientation or combination thereof.

According to some embodiments of the present invention, a hollow, substantially conical, housing flattens and secures the patient's breast and interfaces between the breast and an ultrasound transducer during scanning. The housing applies pressure to the breast to cause compression and somewhat reproducible flattening to allow for scanning with less penetration. The housing secures the breast and fixes the breast in place without causing lateral and/or rotational deformation and/or shearing to the underlying breast tissue. While the housing is fixed to the breast, the housing and the breast are substantially immobile and to not move relative to each other.

Optionally, the housing may also comprise an internal balloon to apply pressure to the breast.

Optionally, the housing may comprise a flexible extender for facilitating ultrasound scanning of substantially large breasts, for example, breasts having a cup size D or larger.

Optionally or alternatively, the housing may comprise a fluid bath in which an ultrasound transducer may operate, thus enhancing the interface between the probe and the breast and/or enabling the probe to interface with the breast at different angles.

According to some embodiments of the present invention, a plurality of ultrasound energy frequencies from concurrent spatial locations may be transmitted into a tissue and/or organ to enhance the images resulting from the ultrasound scan.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

In the following description, components having similar functionality in more than one figure may be referenced in multiple figures by identical reference numerals.

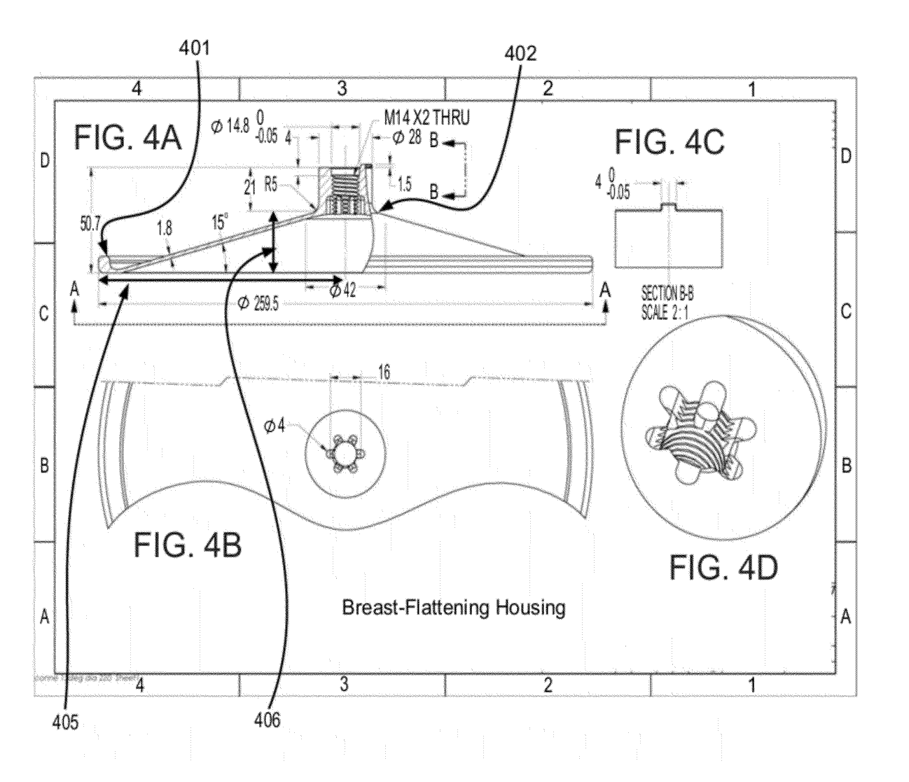

Reference is now also made to FIG. 3 which is a flowchart for using of a breast-flattening housing according to some embodiments of the present invention. As used herein, “breast-flattening housing” means a cone, a dome, a polyhedron, an ellipsoid, and/or any element that is sized and shaped for flattening a breast against the chest wall or other object so that the depth of the breast's tissue is reduced, for example, to less than 4 centimeters. It should be noted that the depth may be higher in substantially large breasts, for example, those having a cup size D or larger. Application of extrinsic pressure on the housing toward the body generates direct substantially uniform linear pressure perpendicular to the surface of the patient's breast, i.e., the pressure applied throughout the breast does not vary by more than 20% throughout the scanned volume. The pressure via the housing secures the breast, fixing it in place, and prevents lateral and/or rotational tissue deformation and/or shearing during scanning; the housing and the breast are substantially immobile relative to each other while the breast is secured within the housing.

As used herein, the phrase “substantially conical housing” means a hollow element configured to contain an organ such as a breast and/or object wherein said element is open at one end for insertion of said organ and/or object, and said element has a shape that resembles a cone, dome, truncated cone, or truncated dome.

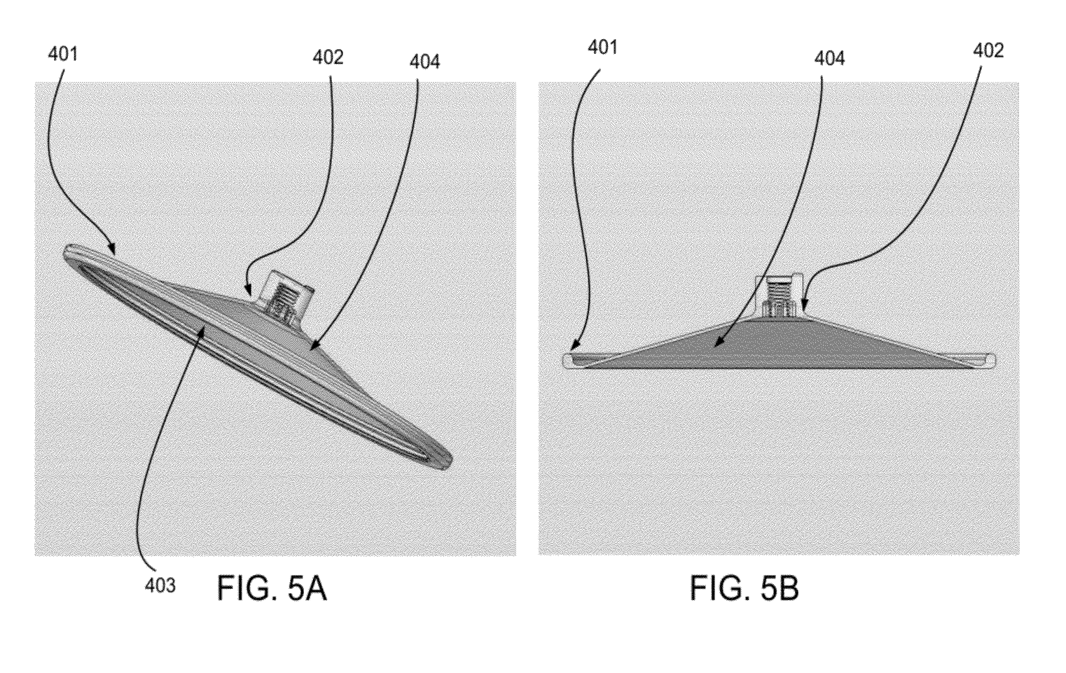

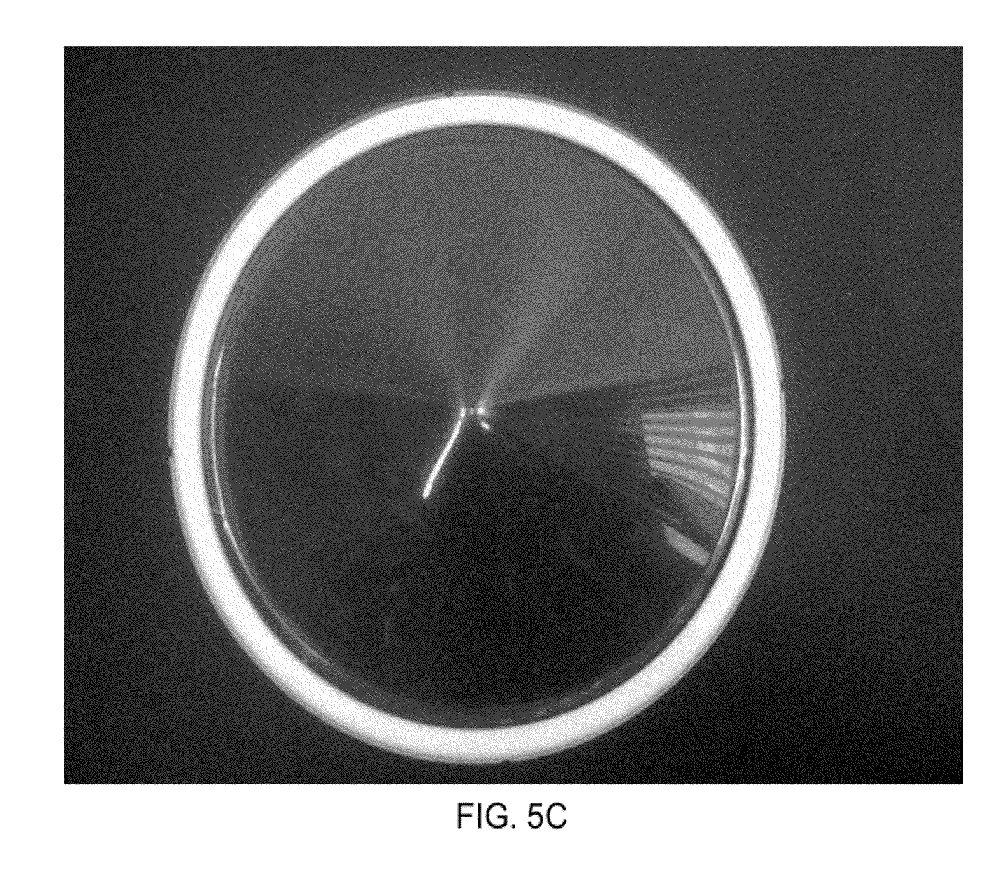

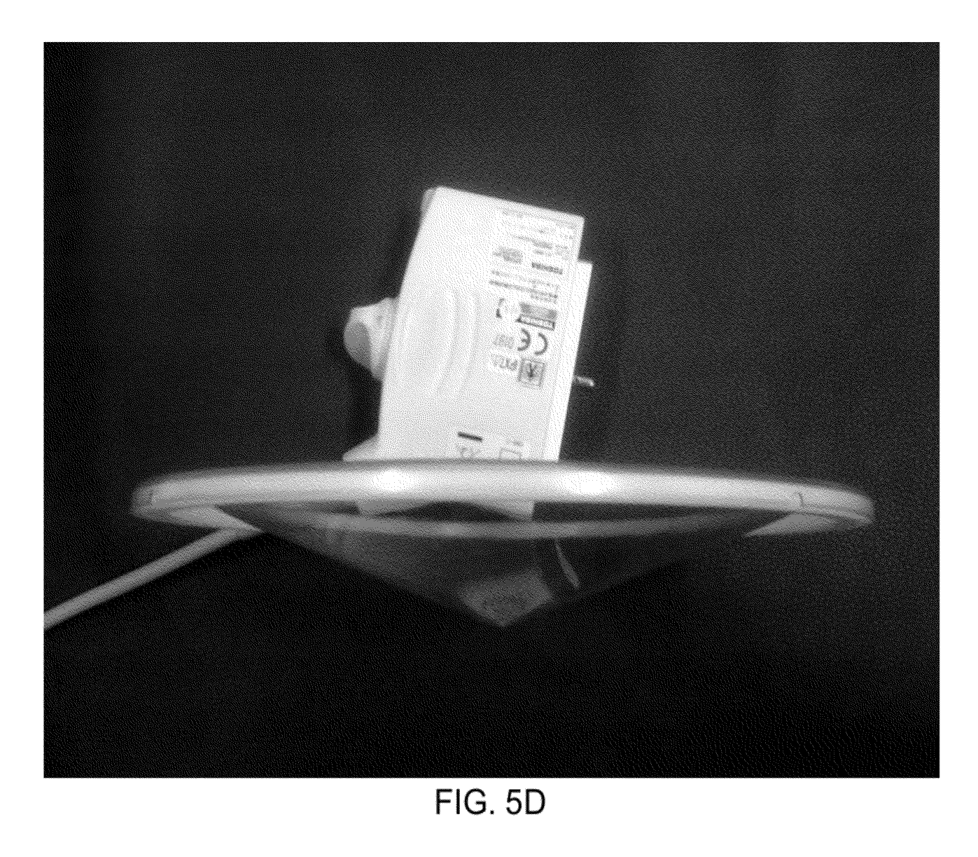

Reference is now also made to FIGS. 4A-4D which are a schematic illustrations of an exemplary breast-flattening housing wherein FIG. 4A is a sectional view through a diameter, FIG. 4B is sectional view of a housing wall at the open end, FIG. 4C is a bottom view of the housing, and FIG. 4D is a view of a threaded element and holes for vacuum application, according to some embodiments of the present invention. Reference is also made to FIGS. 5A-5B which are illustrations of an exemplary breast-flattening housing, according to some embodiments of the present invention and to FIGS. 5C-5D which are photographs of an exemplary breast-flattening housing, according to some embodiments of the present invention. Optionally, the angle between the hollow base 403 and the conical surface 404 as in FIGS. 5A-5B is equal to 15, 17, 20, 25, 40, or 60 degrees, or any intermediate angle. The hollow housing is sized and shaped for covering at least the center of a breast, depending on the size thereof. Optionally, if a breast is of small to medium size, for example, cup size is between A and D, the hollow housing covers the center of the breast and the periphery areas thereof. Optionally, if the breast is large, for example, cup size is larger than D, the hollow housing covers the center of the breast and a portion of the periphery area thereof. Optionally wider hollow housings may be used for covering substantially larger breasts, for example, those having cup sizes which are larger than D. Optionally, the angle between the hollow base 403 and the conical surface 404 as in FIGS. 5A-5B is smaller than 15 degrees. As depicted in FIGS. 4A-4D and FIGS. 5A-5B, the breast-flattening housing may be a substantially truncated conical element having a closed top 402, a substantially hollow base 403 and a conical surface 404. As used herein, “substantially hollow base” means at least 60%, 70%, 80%, 85%, 90%, 95%, and 99% hollow, or any intermediate percentage between the aforementioned values. In an optional embodiment, the ratio between the depth 405 of the conical element and the diameter of the hollow base is 5:8.5 or less. In a preferred embodiment the ratio between the depth 405 of the conical element (approximately 30 millimeters) and the diameter 406 (approximately 260 millimeters) of the hollow base thereof is optionally 3:26 or less, for example as shown in FIG. 4A. Additionally or alternately, the angle between the hollow base 403 and the conical surface 404 is smaller than 30 degrees; the ratio between the depth of the conical element and the diameter of the hollow base thereof is approximately 36:125. In use, an operator, such as a physician or an ultrasound technician, attaches the breast-flattening housing to a scanning device in preparation for performing a scan 305. The housing may comprise a threaded portion at the substantially closed end for connection to the scanning device which may rotate the housing. The patient assumes a position in which the housing and the scanning device may be maneuvered to perform a scan 310. For example, the patient may lie in a supine position, an oblique-supine position, or a prone position. The operator moves the housing to an appropriate location adjacent to the patient and positions the patient's breast in the hollow base of the housing 315. Optionally, vacuum may be applied to the interior of the housing through apertures in the closed end to secure the breast to the housing, for example as described in U.S. patent application Ser. No. 10/559,078, which is incorporated herein by reference. Optionally, vacuum may be applied to the interior of the housing from another location, for example, through an opening near the hollow base. The operator applies pressure to the housing to compress the breast 320 in a direction perpendicular to the surface of the housing so that the depth of the breast's tissue is reduced for example, to less than 4 centimeters. The operator performs the scan 325, maneuvering an ultrasound probe adjacently to the surface of the housing, and at the end of the scanning process the housing is removed from the patient's breast 330.

Optionally, a breast-flattening housing comprises an opening at the substantially closed end through which the nipple of a breast may extend. Vacuum pressure may be applied to the nipple to enhance breast fixation and to enhance coupling of the housing.

Optionally, instead of an ultrasound scanner, a therapeutic device may be used, for example, a high energy therapeutic ultrasound device, an RF energy ablation device, or a cryosurgery device.

In some embodiments of the present invention, the device may comprise an examination bed 901 with a lifting mechanism for height adjustment ranging from 900 mm to 1300 mm and/or a mechanical arm that attaches the device to the examination bed. Optionally, the mechanical arm has four degrees of freedom.

According to some embodiments of the present invention, a device for scanning a patient's breast comprises a mechanical arm that holds and manipulates a scanner. The arm comprises a piston that applies constant pressure to the patient's breast through the scanning device via a housing comprised by the scanning device. The scanning device may orient the housing by rotation before the housing secures the breast. After positioning and orienting the housing and/or the patient's breast, the piston applies constant pressure to fix the housing against the breast and to secure the breast substantially motionless within the housing. Optionally, vacuum may also be applied through the housing to further secure the breast. The mechanical arm pay position and manipulate the device for use while a patient is in prone, supine, or oblique-supine position.

According to some embodiments of the present invention, a region of a surface of a housing is provided with at least one aperture which serves as a window for inserting a diagnosis or treatment tool therethrough. For example, a biopsy tool may be inserted through the aperture. An additional example of treatment via the aperture is brachytherapy. Optionally, the aperture may be sealable. For example, a self-sealable aperture may either be lined with an elastomeric material that allows penetration of the tool and self-seals when the tool is withdrawn or may be covered with a removable, sealed layer that may be peeled off after applying suction to the housing. Additionally or alternatively, the aperture may be manually sealable, for example using a resin and/or other adhesive. The window is situated by manipulating the scanning device with a mechanical arm and/or rotating the housing into position before securing the breast.

According to some embodiments of the present invention, the device may comprise an examination bed 1030 configured to enable scanning while a patient is in a stable prone position. The bed comprises a frame 1031 to which a platform 1033 slidably attached. The platform 1033 is configured with an opening through which a patient's breast may hang dependent. An separate adjustable head support 1032 is slidably attached to the frame 1031 so that the patient's head is supported by the head support 1032 while her body is supported by the platform 1033 and one of her breasts passes through the opening. A scanner 1034 attached to a mechanical arm (not shown) under the examination bed 1030 may be maneuvered to an appropriate location adjacent to the patient's breast, and a housing may compress the patient's breast with pressure provided by a piston comprised by the mechanical arm.

According to some embodiments of the present invention, the device is an electromechanical system for diagnostic B-mode or other modes of ultrasonic imaging of a patient's breast when used with an automatic scanning linear array transducer.

Optionally, Doppler ultrasound imaging is used during the performance of the scan.

Optionally, the device is used for diagnostic or screening imaging scans.

Optionally, the device is used in conjunction with invasive diagnostic procedures, such as biopsy.

Optionally, the device is used for diagnosis and/or in conjunction with treatment of female and/or male patients. Gender references in this document should be considered to relate to both males and females, except where specific anatomical differences make them applicable to only one gender.

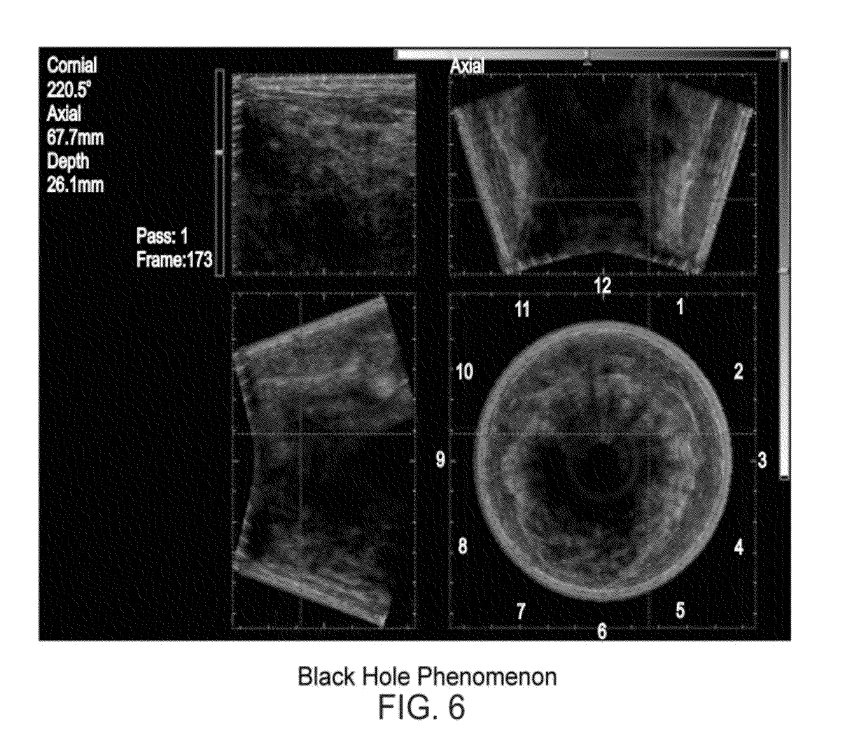

Reference is now also made to FIG. 6 which is an image of an exemplary ultrasound breast scan exhibiting a black hole phenomenon. Ultrasound scanning may be performed when the breast is not flattened or uniformly flattened. When such a scan is performed, breast tissues are not flattened and/or compressed in a manner that allows the ultrasound probe to receive ultrasound beam echoes from all of them. When the ultrasound beam echoes are received from only some of the breast tissues, black holes may be formed in the scan image, reducing the accuracy and usefulness thereof. Henceforth, presence of black holes in ultrasound images due to lack of data is called the black hole phenomenon (BHP). The black hole is caused by data loss of ultrasound beams and shadows created by objects in the breast (Cooper ligaments, cysts, blood vessels, etc.). For example, when high frequency ultrasound beam are used, black holes are formed in a depth greater than about 4 centimeters in a breast as high frequency ultrasound beam echoes seem to be undetectable at such a depth. Generally, localized compression-dependent attenuation is present when scanning frequencies above 10 MHz, but below 6 MHz no such attenuation is seen. This trend does not apply, however, when imaging with harmonics (M. Halliwell, S. Curtis; Visions Magazine 2008 No. 12, page 72). When black holes are formed in a scan, the physician cannot completely and credibly diagnose all the areas in the breast.

Experiments have shown that one contributor to BHP is a lack of breast compression. Use of a breast-flattening housing, for example as described above and depicted in FIGS. 4A-4D and FIGS. 5A-5B, enables fixation of the breast, and may flatten the breast so that the depth of the breast tissue is reduced, for example to less than 4 centimeters, as further described above. Bringing the breast tissue within a given depth, for example, 4 centimeters, eliminates BHP. In an exemplary embodiment of the present invention, a breast-flattening housing may compress breast tissue in a direction substantially perpendicular to the surface of the housing. A breast-flattening housing has a registration pin on the connection, and the breast-flattening housing may be used in a prone position with or without fixation. A circumferential flange 401 may be provided at the open base of the housing to add strength and rigidity. Optionally, the breast-flattening housing is a universal one-size housing for all breast sizes. Optionally, to better accommodate breasts of varying sizes, shapes, and/or densities breast-flattening housings of different sizes are used, for example the distance between the closed end of the housing and the open end of the housing may be 3, 4, or 5 centimeters depth. Optionally, to flatten and compress the volume of a breast, the conical surface may be slanted at an angle between 15 degrees and 72 degrees to a plane concurrent with the open base of the housing. Optionally, a breast-flattening housing may be used with or without applying vacuum pressure to the internal volume of the housing to affix the breast inside the housing. Optionally, the breast-flattening housing is a hollow three-dimensional object that is concave on the inside and convex on the outside whose surface may be a substantially conical, polyhedral, ellipsoidal, domed or other form suitable to secure a human breast substantially without empty space between the breast and the surface of the object.

Another approach to overcoming BHP may be to compress the breast tissues by inflating a balloon inside a housing. The housing may be configured not to flatten the breast, for example, the distance between the closed end of the housing and the open end of the housing may be between 5 and 10 centimeters, or the housing may be a breast-flattening housing similar to the aforementioned breast-flattening housing. This may be accomplished by fabricating one or more balloons into the housing. In some embodiments of the present invention, a housing comprises at least one inner balloon that distends and compresses the breast against the housing internal surface and/or against the chest when the balloon is filled with an ultrasound conducting liquid. A housing comprising one or more balloons may be configured to apply substantially uniform pressure to the breast, or the housing may be configured to apply different pressures to different parts of the breast. The breast may thus be molded during a scan to obtain optimal scanning conditions that may vary according to different physiological and/or pathological conditions. The housing may be placed upon the breast while the balloon is in a deflated state, and ultrasound conducting fluid may be injected into the balloon before a scan in order to inflate the balloon. In such a manner, a scan may be performed while the breast is flattened by the balloon which is in an inflated or partly inflated, state.

Optionally, a balloon may interface between a housing and one side of a breast, for example, a side situated near the center of the chest, so that when inflated, the balloon compresses the breast outward, for example, in the direction of an arm. Alternatively, a balloon may interface between a housing and another side of a breast, for example, a side situated near an arm, to that when the balloon is inflated, the balloon compresses the breast inward, for example, in the direction of the second breast.

In an exemplary embodiment of the present invention, a plurality of balloons may interface between a housing and a breast such that each balloon may be fully or partially inflated at a given time. The inflation and/or deflation of each balloon may be synchronized with ultrasound scanning to perform a plurality of imaging sequences of the breast in different positions without the need to reposition the housing. The inflation and/or deflation may be controlled by a computer, for example, a computer controlling an automatic ultrasound scan, or the inflation and/or deflation may be controlled manually.

Optionally and/or alternatively, a balloon may interface between the entire inner surface of the housing and a breast.

Optionally and/or alternatively, a balloon may occupy space under the apex of the housing interfacing only between the center of the housing and a breast, applying pressure to a nipple and surrounding tissue.

Optionally and/or alternatively, a balloon may be of a torus shape around the inner surface of the housing, applying pressure only around the edges of the breast.

Optionally and/or alternatively, a balloon may be of an irregular shape, applying pressure to the breast in an irregular pattern.

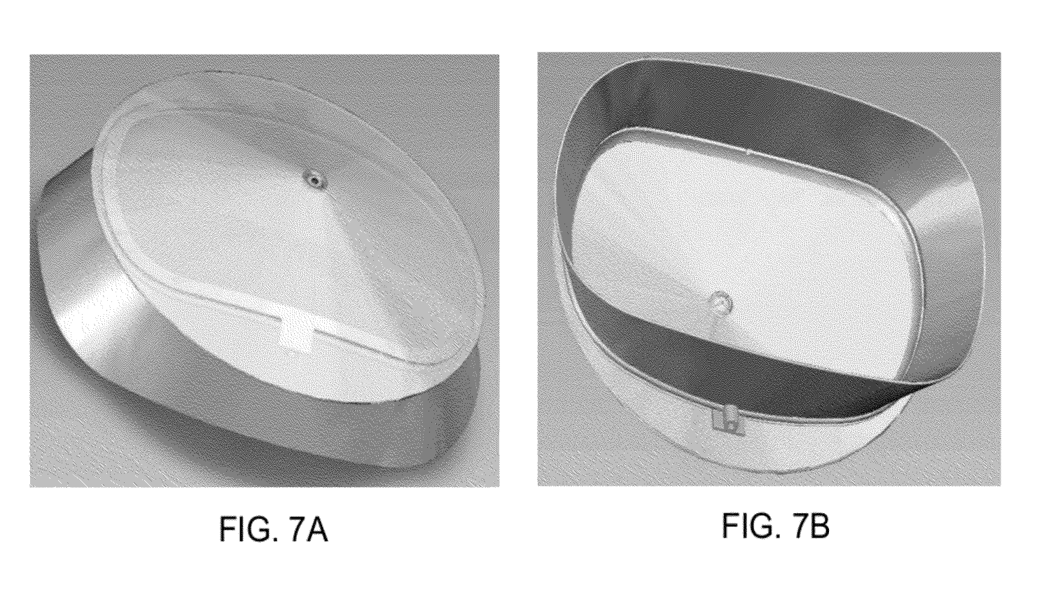

Reference is now also made to FIG. 7A and FIG. 7B which are images of an exemplary a breast-flattening housing with a volume extending attachment, according to some embodiments of the present invention. Scanning with relatively narrow housings may result in a lack of ultrasound data from the axillary section of the breast (breast tail) since this part of the anatomy is not in contact with the housing. Use of some breast-flattening housings may have similar results. In some embodiments of the present invention, a breast-flattening housing covers the breast tail near the patient's shoulder to enable the collection of ultrasound data from said breast tail. In an exemplary embodiment of the present invention, a flexible extender, such as a rubber and/or plastic extender, may be attached to a breast-flattening housing to enable collection of ultrasound data from the breast tail, to aid in housing fixation, to facilitate reproducibility of tests, and/or to avoid requirement of a plurality of housing sizes for use with breasts of different sizes. A housing with an extender may be used while the patient is in either prone or supine position, and it requires vacuum attachment. The extender may be folded to accommodate a plurality of breast sizes. In some embodiments of the present invention, a housing may be elongated to cover the breast tail and to enable collection of ultrasound data from that part of the anatomy.

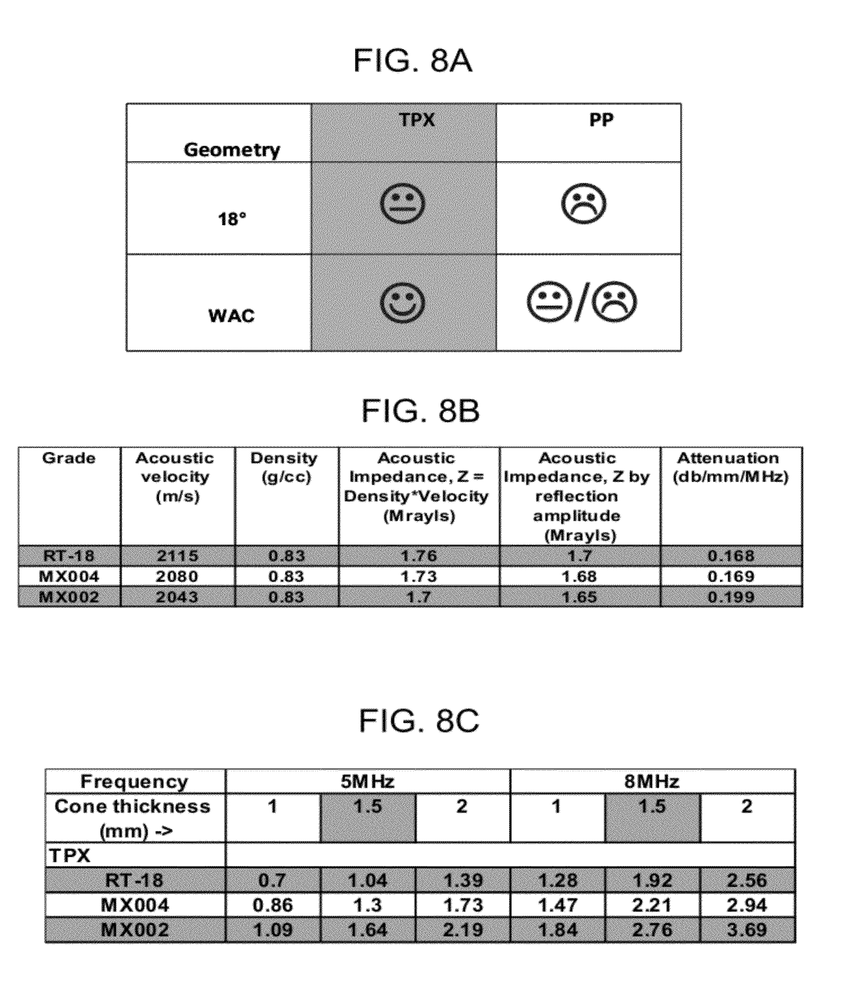

Reference is now also made to FIGS. 8A-8C which are tables providing results of ultrasound scan tests using various housing materials and housing structures wherein the structures are cones. Materials used to manufacture the plastic housing may include polypropylene or polypropene (PP) and/or Polymethylpentene (TPX). PP is a thermoplastic polymer made by the chemical industry and used in a wide variety of applications. Made from the monomer propylene, it is rugged and resistant to many chemical solvents, bases, and acids. TPX is a thermoplastic polymer made of methylpentene monomer units. It is used for autoclavable medical and laboratory equipment. FIG. 8A shows subjective assessments of the results of scans using so-called 18 degree cones (i.e., cones in which the angle between the open base and the conical surface is 72 degrees) and breast-flattening cones (WAC) made of PP and TPX. FIG. 8B is a table showing various acoustic properties of cones made of TPX, and FIG. 8C is a table showing attenuation estimates with acoustic output power (AOP) adjustment (db/RT) for cones made of TPX.

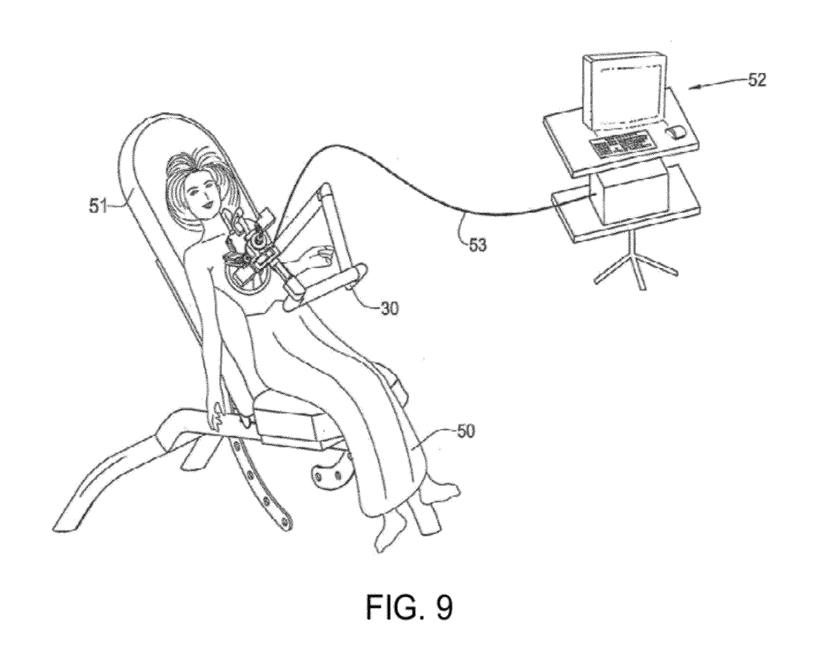

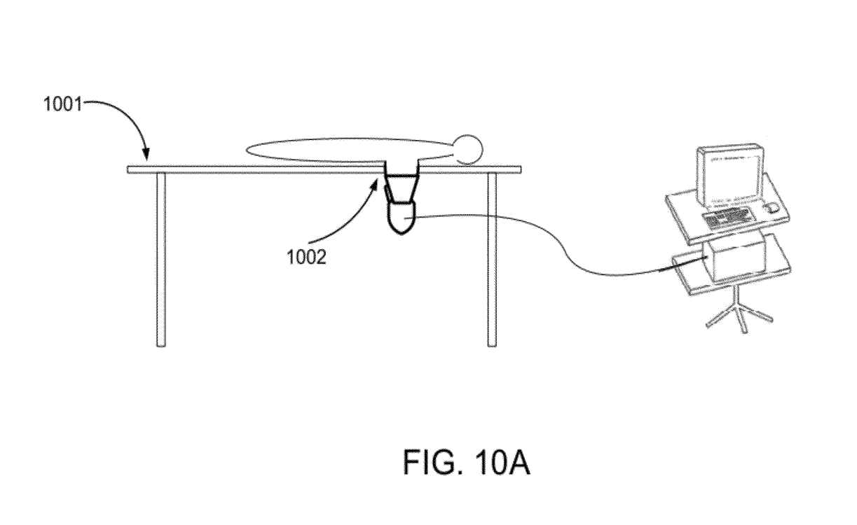

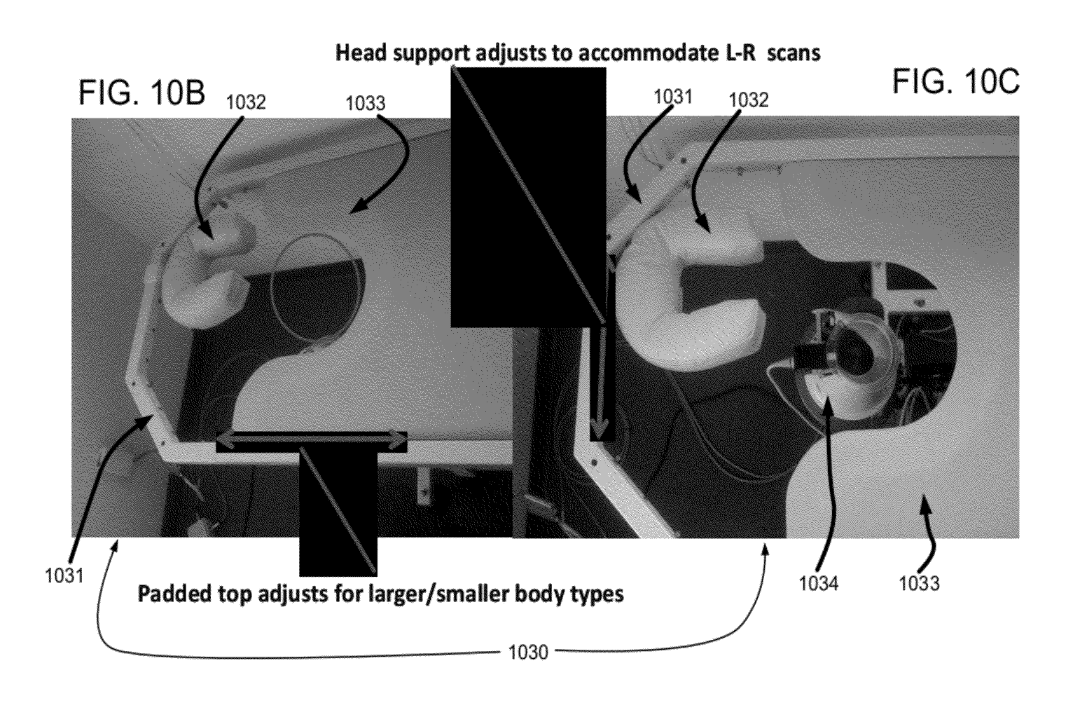

Reference is now also made to FIG. 9 and FIG. 10A which are illustrations, according to optional embodiments of the present invention, representing a patient in oblique-supine and prone positions, respectively. Reference is now also made to FIG. 10B and FIG. 10C which are photographs of a table built to enable access of the device to the patient's breast and/or breasts from below while the patient is in a prone position, according to some embodiments of the present invention. Housings may be used in prone, supine, and/or oblique-supine positions, and they may implement vacuum attachment. The housing may have a registration pin on the connection to assure fixed and repeatable positioning. When the device is used with the patient in a prone position, the patient lies upon a table 1001 constructed with a hole and/or holes 1002 through which the patient's breast and/or breasts may hang pendent such that a housing coupled to a scanning device may be applied to a breast from below the patient's body.

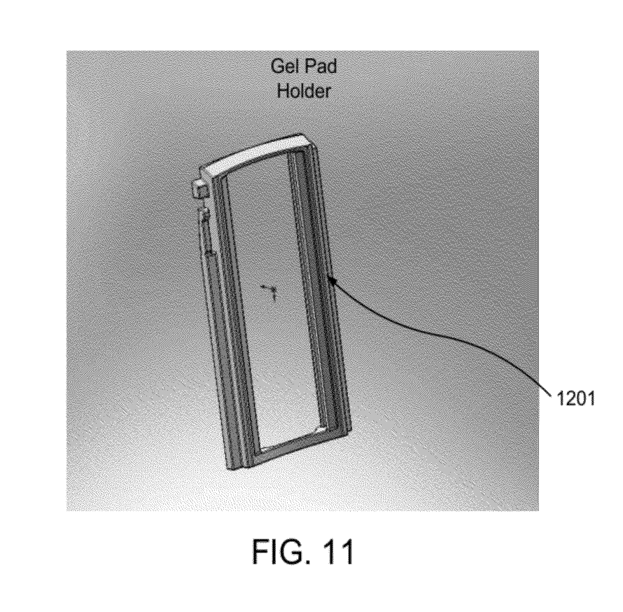

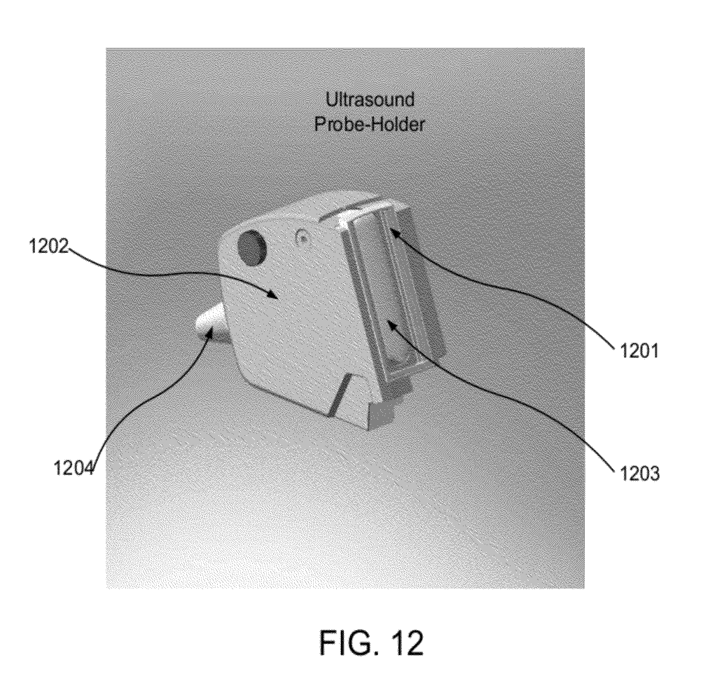

Reference is now also made to FIG. 11 and FIG. 12, which are illustrations respectively representing a gel pad holder 1201 and an ultrasound probe-holder 1202, respectively, according to some embodiments of the present invention. A gel pad 1203 may provide an ultrasonic medium connection between an ultrasound probe 1204 and a housing such as described above. The gel pad 1203 may be a thick, aqueous, flexible, disposable ultrasound standoff for general use and especially for use in difficult to visualize and near field and/or when gels alone are insufficient for an application. Optionally, the gel pad is manufactured by Parker Laboratories, Inc. for medical use. The plastic holder fits onto a probe-holder for an ultrasound probe. Optionally, gel pad thickness is between about 2 to 4 mm thick. Optionally, the gel surface is to be smooth, without knife traces, fingerprints, or scratches to avoid data loss during scanning. Optionally, a gel pad is stored in a sealed container without any gel or water since liquids or gels may change the gel pad structure and/or melt the pad. Optionally, using a disposable gel pad may avoid housing defects, for example a microscopic scratch or leftover gel from a previous procedure that may produce a bubble that causes ultrasound data loss.

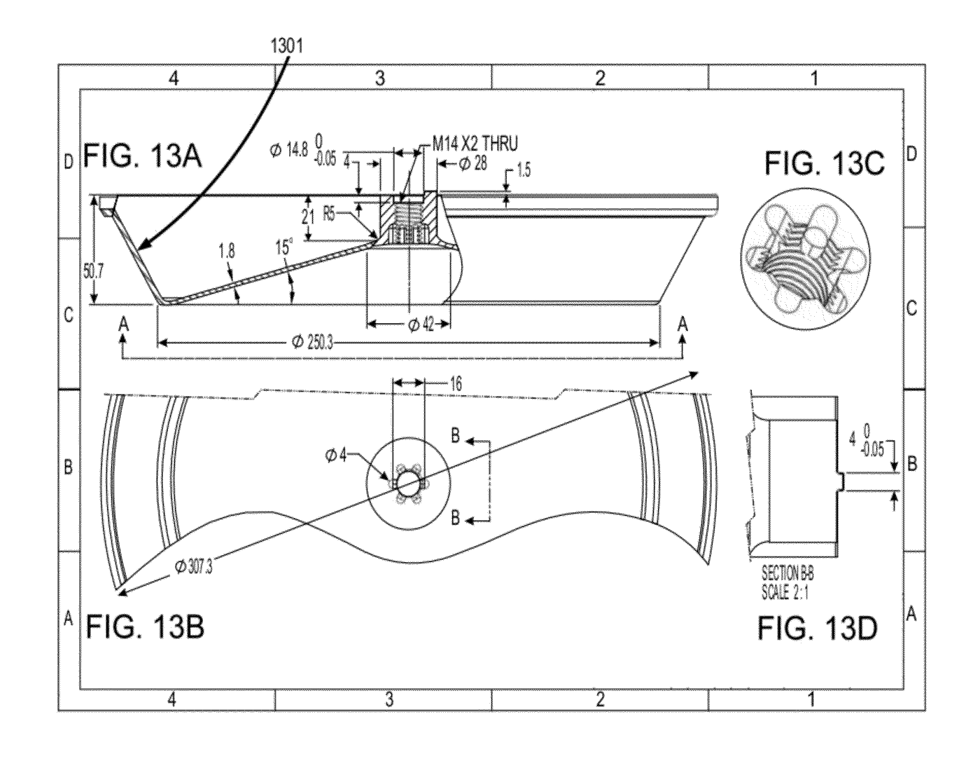

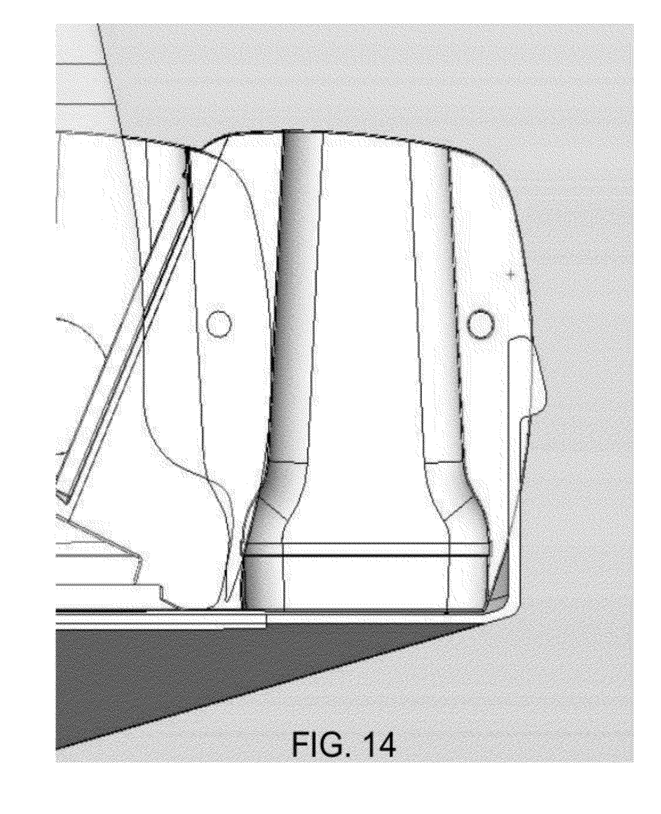

Reference is now also made to FIG. 13 which is a schematic diagram of a fluid-bath comprising a breast-flattening housing for ultrasound imaging, according to some embodiments of the present invention, and to FIG. 14 which is an illustration of a liquid bath used for ultrasound imaging, according to some embodiments of the present invention. In some embodiments of the present invention, a probe may be moved adjacent to breast-flattening housing in a liquid bath and/or changing the probe's angle relative to the housing. The aforementioned configuration provides for a continuous ultrasound conducting interface between the probe and the breast. The probe may thus be used at angles relative to the breast that cannot be achieved without lifting the probe face from the surface of the housing or breast, hence losing data in the absence of such a conducting interface. Directional beam steering may acquire data from multiple angles simultaneously. Changing the probe's angle may bypass intervening anatomical structures and thus mitigate shadows in the ultrasound images. The liquid bath itself comprises a breast-flattening housing (as described above) with high shoulders 1301 that contains an ultrasound conducting material, for example, water or ultrasound gel wherein the ultrasound conducting material covers the outer surface of the breast-flattening housing. Using this housing is only possible in a supine position, and vacuum attachment may or may not be used.

According to some embodiments of the present invention, multiband ultrasound transmission is used. Frequency and harmonic imaging seem to strongly influence localized compression dependent ultrasound attenuation which leads to shadows and image degradation. Typical probe frequency is between 5-12 MHz. Using fundamental frequency imaging, localized attenuation is observed in uncompressed breasts at frequencies above 10 MHz, but is not seen below 6 MHz. Using harmonic frequency imaging, such attenuation is seen at frequencies of 14, 12, and 6 MHz (M. Halliwell, S. Curtis; Visions Magazine 2008 No. 12, page 74). The probe may transmit ultrasound signals from a plurality of spatial locations into a patient's breast such that a plurality of ultrasound frequencies is transmitted from one or more of said spatial locations. The information obtained from received scattered ultrasound signals may then be analyzed to create two-dimensional and/or tomographic images representing the scanned tissue. It should be noted that higher frequency ultrasound signals produce higher quality images than lower frequency ultrasound signals, however, lower frequency ultrasound signals penetrates tissues to a greater depth than higher frequency ultrasound signals. In an exemplary embodiment of the present invention, a housing secures a breast for scanning, and multiple ultrasound scanning sequences are performed in a series, each sequence using a different ultrasound frequency, for example, 12 MHz, 10 MHz, 8 MHz, and 6 MHz. Individual images from scanning sequences in the series are combined to produce enhanced compound images. Additionally or alternatively, different ultrasound frequencies are interleaved within a single series.

It is expected that during the life of a patent maturing from this application many relevant ultrasound scanning systems will be developed and the scope of the term ultrasound scanning systems is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A device configured for performing a spatial scan of a patient's breast, comprising:
a substantially hollow housing having a substantially conical surface between a top and a substantially hollow base, said substantially hollow housing being sized and shaped for covering at least the center of the breast, said substantially hollow housing maintains a fixed conical shape when being pressed against the breast;
a scanning device configured for performing a scan while being maneuvered in a scanning pattern adjacent to said substantially conical surface while said substantially hollow housing substantially remains in place to fixate the breast;
a securing unit for securing the substantially hollow housing to the breast during said scan so that the breast being substantially fixed relative to the housing; and
wherein a distance between said top and said substantially hollow base is less than 5 centimeters.

2. The device of claim 1, wherein said housing is substantially domed, conical, polyhedral, or ellipsoidal.

3. The device of claim 1, wherein the ratio between said distance and a diameter of said substantially hollow base is less than 5:8.5.

4. The device of claim 3, wherein the ratio between said distance and a diameter of said substantially hollow base is less than 3:26.

5. The device of claim 1, wherein said substantially hollow housing is sized and shaped for applying substantially uniform extrinsic direct linear pressure perpendicular to at least 80 percent of the surface of the patient's breast.

6. The device of claim 1, wherein said substantially conical surface is slanted at an angle of less than 30 degrees to a plane concurrent with said substantially hollow base.

7. The device of claim 1, wherein the distance between said top and said substantially hollow base is less than 3 cm.

8. The device of claim 1, wherein holes penetrate said substantially conical surface through which vacuum pressure is applied inside said substantially hollow housing.

9. The device of claim 1, further comprising a flexible extender affixed to said housing adjacent to said substantially hollow base to increase the volume of said housing.

10. The device of claim 1, wherein said housing further comprises a liquid bath in which said scanning device performs a spatial scan of a patient's breast.

11. The device of claim 10, wherein said scanning device is positionable at a plurality of angles relative to said housing.

12. The device of claim 1, wherein said housing comprises an inflatable balloon inside said substantially hollow housing, said balloon being configured to compress the tissue of said breast in an inflated configuration.

13. The device of claim 1, wherein said housing consists essentially of polymethylpentene.

14. The device of claim 1, wherein said device comprises a mechanical arm having at least 4 degrees of freedom.

15. The device of claim 14, wherein said mechanical arm further comprises a piston configured to compress a breast of a patient during scanning.

16. The device of claim 1, further comprising a table for enabling a patient to lie in a stable prone position, wherein said table enables the patient's breast to hang dependent through an opening in said table, and said securing unit secures the breast during said scan.

17. A device configured for performing a spatial scan of a patient's breast, comprising:
a substantially hollow housing having a substantially conical surface between a top and a substantially hollow base;
a scanning device configured for performing a scan while being maneuvered adjacently to said substantially conical surface; and
an inflatable balloon positioned in said substantially hollow housing and configured for being inflated by a ultrasound conducting liquid before said scan.

18. The device of claim 17, wherein said housing further comprises an aperture sized and shaped for facilitating insertion of a tool for at least one of diagnosis and treatment.

19. A method of performing ultrasound imaging comprising;
transmitting a plurality of distinct ultrasound frequencies into breast tissue;
intercepting scattered signals of said distinct ultrasound frequencies to create a plurality of images from said breast tissue; and
combining said plurality of images to create an image representing said breast tissue.

20. The method of claim 19, wherein said imaging comprises tomography.

21. A method of performing ultrasound imaging comprising;
a) placing the patient's breast into a substantially hollow housing and securing said breast therein, said substantially hollow housing maintains a fixed conical shape when being pressed against the breast;
b) compressing the tissue of said breast such that the depth of said tissue from the top to the base of said housing is less than 5 centimeters; and
c) scanning the breast with a scanning device while said substantially hollow housing substantially remains in place to fixate the breast.

22. The method of claim 21, wherein said scanning comprises maneuvering said scanning device adjacent to said housing in a substantially rotational or helical scanning pattern.

23. The method of claim 21, wherein said compressing comprises inflating a balloon that is enclosed in said housing with ultrasound conducting liquid.

24. The method of claim 21, further comprising filling a bath with ultrasound-conducting liquid, submerging said scanning device in said liquid and scanning said breast while said scanning device is submerged in said liquid.

25. The method of claim 24, further comprising positioning said scanning device at a plurality of angles relative to said breast.

26. The method of claim 21, further comprising affixing, configuring, and aligning a flexible extender to said housing to modify the area of said breast that is available for scanning.

27. The method of claim 21 wherein said securing comprises the application of vacuum pressure to the inside of said housing through holes in the surface of said housing.

28. A method for scanning a body organ, comprising:
providing a probe;
mounting a housing around a body organ;
positioning a container comprising an ultrasound conducting material between said housing and said probe; and
maneuvering said probe adjacently to said housing for performing a scan of at least a portion of said body organ while said ultrasound conducting material being in physical contact with said housing and said probe.

29. The method of claim 28, wherein said container is a bath for holding said ultrasound conducting material;
said mounting comprising fully or partially submerging said probe in said ultrasound conducting material.

30. The method of claim 28, wherein said container is a holder containing a replaceable gel pad, and said positioning comprises attaching said gel pad to said probe, such that during said scanning said pad physically interfaces between said probe and said housing.
